(19) United States
(12) Patent Application Publication
(10) Pub. No.: US 2005/0142548 A1
(43) Pub. Date: Jun. 30, 2005

(54) Allelic variants of grp50

(76) Inventors: Anne Margaret Thomson, Scotland (GB); Donald, Robert Dunbar, Scotland (GB)

Correspondence Address: AKZO NOBEL PHARMA PATENT DEPARTMENT, PO BOX 318, MILLSBORO, DE, 19966, US

(21) Appl. No.: 10/484,096

(22) PCT Filed: Jul. 8, 2002
(86) PCT No.: PCT/EP02/07639

(30) Foreign Application Priority Data
Jul. 13, 2001 (EP) 01202690.2

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

The present invention provides isolated polynucleotides encoding a receptor gene called GPR50 having at least one polymorphic sites. It furthermore provides a method for analysing polimorphic sites in said receptor gene. Certain of these polynucleotides having a polymorphic site (allelic variants) are found to be more prevalent in a population of patients with clinical Bipolar Depression compared to a control population. A method for the genetic testing of Bipolar Depression is a further embodiment of the present invention. Furthermore, polynucleotides encompassing these polymorphic sites, the invariant distal or proximal to the polymorphic site localized polynucleotides as well as the polynucleotides encoding GPR50 are part of the invention. The present invention also provides a recombinant cell line expressing these novel receptors at appropriate levels such that novel compounds active at these receptors may be identified for therapeutic use.

The present invention provides isolated polynucleotides encoding a receptor gene called GPR50 having at least one polymorphic site. It furthermore provides a method for analysing polymorphic sites in said receptor gene. Certain of these polynucleotides having a polymorphic site (allelic variants) are found to be more prevalent in a population of patients with clinical Bipolar Depression or Unipolar Depression compared to a control population. A method for the genetic testing of Bipolar Depression and Unipolar Depression is a further embodiment of the present invention. Furthermore, polynucleotides encompassing these polymorphic sites, the invariant distal or proximal to the polymorphic site localized polynucleotides as well as the polynucleotides encoding GPR50 are part of the invention.

The present invention also provides a recombinant cell line expressing these is 5 novel receptors at appropriate levels such that novel compounds active at these receptors may be identified for therapeutic use.

G-protein-coupled receptors (GPCRs) are a large superfamily of membrane receptors that transduce a wide array of extracellular signals into intracellular responses. Stimulation of a receptor by its cognate ligand leads to activation of an associated heterotrimeric G protein, which in turn regulates intracellular pathways which have an effect on effector enzymes and ion channels (Wess et al., 1997). Some examples of endogenous ligands which bind to GPCRs include neurotransmitters, neuropeptides, hormones, chemokines and odorants. This receptor family is therefore involved in the regulation of multiple physiological processes which encompass neurotransmission, feeding, mood, pain, reward, vision and smell, as well as inflammatory and immune responses (Strader et al., 1995).

GPCRs have a proven history as excellent therapeutic targets with between 40-50% of drug targets to date being GPCRs (Murphy et al., 1998). The GPCR family comprise over 350 cloned human members but only some of the endogenous ligands for these receptors have been identified. There are an increasing number of G-protein-coupled receptors which are being identified by molecular cloning methods and bioinformatics for which the physiological ligands are not known; these are referred to as orphan receptors. Many of these orphan GPCRs are expressed in the brain, and therefore may represent novel therapeutic targets for the treatment of CNS disorders (O'Dowd et al., 1997).

Reverse pharmacology or functional genomics is currently being adopted within the drug discovery process. This is gene-based biology which aims to pharmacologically validate novel genes by either identifying surrogate ligands is or their endogenous ligand.

There is evidence to suggest that in addition to novel orphan GPCRs, there also exist novel GPCR gene sub-families that bind previously unidentified ligands. Because many orphan GPCRs await to be assigned a natural ligand, many of these receptors may bind novel ligands which have not thus far been identified (Civelli et al., 1999).

Orphan GPCRs are predicted to bind ligands, as it is postulated that inactive receptors should have been evolutionary discarded. Orphan receptors may therefore be used as baits to isolate their natural ligands or surrogate ligands. The use of this strategy in identifying novel ligands is exemplified in the identification of orphanin/nociceptin, orexins/hypocretins and prolactin-releasing peptide (Reinscheid et al., 2000, Sakurai et al., 1998, and Hinuma et al., 1998).

Many known G protein coupled receptors (GPCRS) are well established drug targets with a significant number of currently available drugs targeting such GPCRs (Wilson et al., 1998). Following activation of a GPCR by ligand binding to the receptor, the signal is amplified through a range of signal transduction cascades and consequently, regulation of this signal transduction pathway via a ligand binding to a GPCR offers the facility to modulate a tightly controlled biological pathway.

GPCRs mediate a wide range of biologically relevant processes and are responsive to a wide variety of stimuli and chemical/neurotransmitters, including light, biogenic amines, amino acids, peptides, lipids, nucleosides, and large polypeptides. How the cloning of a particular receptor has led to the development of a therapeutic compound is particularly exemplified in the case of the serotonin and adrenergic receptors. Additionally, a number of diseases are reported to be associated with mutations in known GPCRs (Wilson et al., 1998). The signaling pathways that mediate the actions of GPCRs have also been implicated in many biological processes significant to the pharmaceutical industry. Such signaling pathways involve G proteins, second messengers such as cAMP or calcium), effector proteins such as phospholipase C, adenylyl cyclase, RGS proteins, protein kinase A and protein kinase C (Lefkowitz, 1991).

For example a GPCR can be activated by a ligand binding to the receptor resulting in the activation of a G protein which conveys the message onto the next component of the signal transduction pathway. Such a component could be adenylyl cyclase. In order for activation of this enzyme, the relevant G protein, of which there is a family, must exchange GTP for GDP, which is bound when the G protein is in an inactive state. The exchange of GDP for GTP occurs following the binding of ligand to the GPCR, however, some basal exchange of GDP for GTP can also occur depending on the receptor under investigation.

The conversion of GTP bound at the G protein to GDP occurs by hydrolysis and is catalysed by the G protein itself. Following this hydrolysis the G protein is returned to its inactive state. Consequently, the G protein not only mediates the transfer of the signal from the activated receptor to the intracellular signaling pathway, but also introduces an additional level of control, by controlling the length of time which the receptor can activate the intracellular signaling pathway through the GTP bound G protein.

In general the topology of these receptors is such that they contain 7 transmembrane (TM) domains consisting of approximately 20-30 amino acids. Consequently, these receptors are frequently known as 7TM receptors. These 7TM domains can be defined by consensus amino acid sequences and by structural prediction algorithms such as the Kyte Doolittle programme (Probst et al., 1992). Within the putative transmembrane domains, hydrophobic helixes is are formed which are connected via extracellular and intracellular loops. The N-terminal end of the polypeptide is on the exterior face of the membrane with the C-terminal on the interior face of the membrane.

A number of additional features are frequently observed in GPCRs. These include glycosylation of the N-terminal tail. A conserved cysteine in each of the first two extracellular loops, are modified such that disulphide bonds are formed, which is believed to result in a stabilised functional tertiary structure. Other modifications which occur on GPCRs include lipidation (e.g. palmityolation and farnesylation) and phosphorylation. Phosphorylation events often occur in the third intracellular loop and in the C-terminal cytoplasmic tail of GPCRs. G protein coupled receptor kinases (GRKs) are known to phosphorylate GPCRs on multiple sites with threonine and serine residues as targets. These phosphorylation events are important for regulating receptor internalisation, desensitisation, and/or downregulation pathways (Tsao and Zastrow, 2000; Tiruppathi et al., 2000; Jackson et al., 2000). Consequently, specific mutations in particular regions of the GPCR can have functional significance on downstream intracellular signaling events.

Bacteriorhodopsin is a 7TM GPCR found in the microorganism Halobacterium salinarum. This bacterium uses light as its sole source of energy and the protein bacteriorhodopsin serves as a light-driven proton pump to transport protons across the cell membrane. Bacteriorhodopsin is therefore often used as a simple model to study some of the structure/function characteristics of the more complex mammalian GPCRs. The crystal structure of bacteriorhodopsin has recently been solved (Kuhlbrandt, 2000; Palczewski et al., 2000), and therefore it can serve as a structural template for other GPCRs including the assignment of secondary structural elements and the location of highly conserved amino acids. Rhodopsin is intermediate in size among members of the GPCR family and thus can feature most of the essential parts of functional importance in G-protein activation. The lengths of the seven transmembrane helices and of the three extracellular loops are expected to be nearly the same for most of the family members.

In addition to activating intracellular signaling pathways, GPCRs can also couple via G proteins to additional gene families such as ion channels, transporters and enzymes. Many GPCRs are present in mammalian systems exhibiting a range of distribution patterns from very specific to very widespread. For this reason, following the identification of a putative novel GPCR, assigning a therapeutic application to the novel GPCR is not obvious due to this diverse function and distribution of previously reported GPCRs.

There is clearly a need to identify and characterize novel GPCRs that can function to alter disease status either correction, prevention or amelioration. Such diseases are diverse and include, but are not exclusive to, depression, schizophrenia, anxiety, neurological disorders, obesity, insomnia, addiction, neurodegeneration, hypotension, hypertension, acute heart failure, atherothrombosis, atherosclerosis, osteoporosis, rheumatoid arthritis and infertility.

The present invention provides novel allelic variants for the G-protein-coupled receptor termed GPR50 or melatonin receptor-related receptor (MRR). GPR50 is an orphan GPCR that displays most sequence similarity to the cloned MeI1a and MeI1b melatonin receptors (Reppert et al., 1996). Although the MeI1a and MeI1b receptors have each been shown to bind [¹²⁵I]Iodomelatonin with high affinity, GPR50 was found not to bind this hormone in ligand binding studies following transient transfection of the receptor into COS-7 cells (Reppert et al., 1996; Conway et al., 2000, Gubitz and Reppert, 2000). Melatonin is the main hormone secreted from the pineal gland which modulates the timing of circadian rhythms and may be involved in mood regulation (Reppert et al., 1995).

Human GPR50 mRNA is expressed in pituitary and hypothalamus and in-situ hybridisation experiments have demonstrated it to be heterogeneously distributed in pituitary and to be localised in infundibular stalk and mediobasal hypothalamus (Reppert et al., 1996). Drew et al recently provided evidence that the expression of GPR50 in regions of the hypothalamus and the epithelial layer lining the 3rd ventricle and the paraventricular thalamic nucleus is conserved between mouse, rat and hamster (Drew et al., 2001). These data indicate an important physiological role for the receptor. Furthermore, using in situ data we have found discrete expression of GPR50 in several nuclei of the hypothalamus and additionally in the hippocampus. Thus, GPR50 expression appears to be limited to regions of the brain associated with the HPA axis that may be implicated in depression, schizophrenia and anxiety.

The various forms of depression are defined and are separately diagnosed according to criteria given in handbooks for psychiatry, for example in the Diagnostic and Statistical Manual of Mental Disorders 4th edition (DSM-IV) published by the American Psychiatric Association, Washington, D.C. (1994).

The human GPR50 gene is X-linked and is localised to Xq28 (Gubitz and Reppert, 1999). The loci of over 20 genetic disorders have been found to converge on this gene-rich chromosome region, therefore making GPR50 a possible candidate gene for such diseases.

Bipolar affective disorder (BPAD) is a psychiatric illness which shows a combination of depression and elevated mood in cycles, and this disease has been demonstrated to have linkage to the Xq28 locus (Baron et al., 1994; Stine et al., 1997). No previous genetic studies have indicated that GPR50 is associated with psychiatric disease.

According to the present invention, several polynucleotides have been identified comprising polymorphic sites on the GPR50 gene. These are called allelic variants of GPR50. These allelic variants might help to understand the mechanisms of inheritance of psychiatric disorders, preferably BPAD or Unipolar Depression (UP). The polynucleotides or parts thereof might furthermore be used in genetic testing of these disorders. The polynucleotides parts are preferably at least 10 contiguous nucleotides, preferably 10-100 nucleotides. They can be used in hybridisation-based nucleic acid detection methods. It will be clear that the fragments comprising part of the sequence as obtained from SEQ ID NO: 1 can be used for this purpose as well as fragments comprising the allelic variant sequence.

The object of the present invention is to provide a polynucleotide comprising the whole sequence encoding the GPR50 precursor protein or the mature protein comprising an allelic variant. Also the complete mRNA sequence or the genomic sequence of GPR50 form part of the invention provided that the sequence has at least one polymorphic site deviating from the sequence as identified in SEQ ID NO: 1. The most preferred polymorphic sites are located at positions 1582, 1804 and 1503-1504. Preferably the polynucleotide has an A or G at position 1582 and/or 1804, and/or an insertion/deletion at position 1503-1504. The insertion at nucleotide position 1503-1504 preferably consists of 12 nucleotides, more preferably the nucleotide stretch ACC ACT GGC CAC. The strongest association with BPAD and UP is the absence of the insertion at position 1503-1504 and/or the polymorphic site at position 1804. Preferably this site bears the nucleotide A.

To accommodate codon variability, the invention also includes sequences coding for the same amino acid sequences as the sequences disclosed herein. The nucleotide sequence of SEQ ID NO: 1 encodes a protein the sequence of which is indicated in SEQ ID NO: 9. The invention therefore also includes polynucleotide sequences encoding the protein of SEQ ID NO: 9 with the provison that the nucleotide sequences comprise polymorphic sites according to the invention. Also portions of the coding sequences coding for individual domains of the expressed protein are part of the invention. Sometimes, a gene is expressed in a certain tissue as a splicing variant, resulting in an altered 5′ or 3′ mRNA or the inclusion of an additional exon sequence. These sequences as well as the proteins encoded by these sequences all are expected to perform the same or similar functions and form also part of the invention.

The sequence information as provided herein should not be so narrowly construed as to require inclusion of erroneously identified bases. The specific sequence disclosed herein can be readily used to isolate the complete genes which in turn can easily be subjected to further sequence analyses thereby identifying sequencing errors.

Thus, the present invention provides for isolated polynucleotides encoding GPR50 allelic variants.

The DNA according to the invention may be obtained from cDNA. The tissues preferably are from human origin. Preferably ribonucleic acids are isolated from pituitary, hypothalamus or other tissues. Alternatively, the coding sequence might be genomic DNA, or prepared using DNA synthesis techniques. The polynucleotide may also be in the form of RNA. If the polynucleotide is DNA, it may be in single stranded or double stranded form. The single strand might be the coding strand or the non-coding (anti-sense) strand. Small fragments can easily be prepared using well-known chemical synthesis techniques.

The present invention further relates to polynucleotides allelic variants of SEQ ID NO: 1 having slight variations. Polynucleotides having slight variations encode polypeptides which retain the same biological function or activity as natural, mature allelic forms of the protein. Alternatively, also fragments of the above mentioned polynucleotides which code for domains of GPR50 protein which still are capable of binding to targets are embodied in the invention.

Such polynucleotides can be identified by hybridisation under preferably highly stringent conditions. According to the present invention the term “stringent” means washing conditions of 1×SSC, 0.1% SDS at a temperature of 65° C.; highly stringent conditions refer to a reduction in SSC towards 0.3×SSC, more preferably 0.1×SSC.

Thus also derivatives of the polynucleotides are part of the invention. Under the term derivative is to be understood any polynucleotide encoding GPR50 allelic variants having at least one polymorphic site and which have at least 90%, preferably 95% and more preferably 98% and even more preferably at least 99% identity with SEQ ID NO: 1. Such polynucleotides encode polypeptides which retain the same biological function or activity as the natural, mature allelic forms of the protein. The allelic variations preferably are located at the above identified sites at positions 1582, 1804 and 1503-1504 of SEQ ID NO:1. Preferably the polynucleotide has an A or G at position 1582 and/or 1804, and/or an insertion at position 1503-1504. The insertion at nucleotide position 1503-1504 preferably consists of 12 nucleotides, more preferably the nucleotide stretch ACC ACT GGC CAC.

The percentage of identity between two sequences can be determined with programs such as DNAMAN (Lynnon Biosoft, version 3.2). Using this program two sequences can be aligned using the optimal alignment algorithm (Smith and Waterman, 1981). After alignment of the two sequences the percentage identity can be calculated by dividing the number of identical nucleotides between the two sequences by the length of the aligned sequences minus the length of all gaps.

Another aspect of the invention relates to polynucleotides having a nucleotide sequence capable of specifically hybridizing to the invariant proximal or invariant distal nucleotide sequence of a polymorphic site of SEQ ID NO: 1, and being used to specifically detect the single nucleotide polymorphism site. Such polynucleotides are especially useful in assays based on primer elongation methods such as e.g. PCR.

It is a further object of the present invention to provide a method for analyzing polynucleotides from an individual and determine a nucleotide occupying a polymorphic site of SEQ ID NO: 1. Preferably the nucleotides at positions 1503-1504, 1582 and 1804 are to be determined. It has been found that at is nucleotide position 1503-1504 an insert might be present, preferably of 12 nucleotides, more preferably the nucleotide stretch ACC ACT GGC CAC. Nucleotide positions 1582 and 1804 are preferably occupied by A or G. Polymorphic variants comprising combinations of these variants have been found by sequencing nucleic acids form several individuals. The seven possible allelic variants for GPR50 are listed (SEQ ID NO: 2 to 8 for nucleotide sequence and SEQ ID NO: 10 to 16 for amino acid sequence).

The invention thus relates to the use of the GPR50 gene as part of a diagnostic assay for psychiatric disorders related to mutations in the nucleic acid sequences encoding this gene. Such mutations may e.g. be detected by using PCR (Saiki et al., 1986) or specific hybridisation. Also the relative levels of RNA can be determined using e.g. hybridisation or quantitative PCR technology or DNA microarrays.

The presence and the levels of the GPR50 receptor itself can be assayed by immunological technologies such as radioimmuno assays, Western blots and ELISA using specific antibodies raised against the receptor. Such techniques for measuring RNA and protein levels are well known to the skilled artisan.

The determination of expression levels of the receptors in individual patients may lead to fine tuning of treatment protocols.

All of the polynucleotides according to the present invention are contained in the cytoplasmic tail of this receptor. The C-terminal tail of GPCRs has been reported to differentially dictate receptor downregulation, internalisation and/or desensitisation pathways (Tsao and Zastrow, 2000; Trapaidze et al., 2000; Wang et al., 2000). The polynucleotides provided here introduce threonines in the C-terminal tail of GPR50. GRKs are known to phosphorylate GPCR C-terminal tails at serine and threonine residues and this has been shown to result in receptor desensitisation. Certain GPR50 allelic variants might alter desensitisation, therefore having a significant effect on the functionality of this receptor.

In another aspect of the invention, there is provided a polypeptide comprising the amino acid sequence encoded by the above described DNA molecules.

Preferably, the polypeptide according to the invention comprises variants of at least part of the amino acid sequences as shown in SEQ ID NO: 9 with amino acid substitutions at positions 528 and/or 602 and/or insertions at positions 501-502. Preferred variants are polypeptides comprising Thr or Ala at amino acid position 528, and/or lIe or Val at position 602, and/or an insertion at position 501-502. The position refers to the amino acid sequence in SEQ ID NO: 9. The most preferred insertion is Thr-Thr-Gly His.

Also functional equivalents, that is polypeptides homologous to the variants of SEQ ID NO: 9 or parts thereof having variations of the sequence while still maintaining functional characteristics, are included in the invention.

The functional equivalent variations that can occur in a sequence may be demonstrated by (an) amino acid difference(s) in the overall sequence or by deletions, substitutions, insertions, inversions or additions of (an) amino acid(s) in said sequence. Amino acid substitutions that are expected not to essentially alter biological and immunological activities, have been described. Amino acid replacements between related amino acids or replacements which have occurred frequently in evolution are, inter alia Ser/Ala, Ser/Gly, Asp/Gly, Asp/Asn, Ile/Val (see Dayhof, M. D., Atlas of protein sequence and structure, Nat. Biomed. Res. Found., Washington D.C., 1978, vol. 5, suppl. 3). Based on this information Lipman and Pearson developed a method for rapid and sensitive protein comparison (Lipman and Pearson, 1985) and determining the functional similarity between homologous polypeptides.

The polypeptides according to the present invention include the polypeptides comprising the allelic variants of SEQ ID NO: 9 but also their derivatives, i.e. polypeptides with a similarity of 80%, preferably 90%, more preferably 95%, even more preferably 98% as compared to SEQ ID NO: 9. Also portions of such polypeptides still capable of conferring biological effects are included. Especially portions which still bind to ligands form part of the invention. Such portions may be functional per se, e.g. in solubilised form or they might be linked to other polypeptides, either by known biotechnological ways or by chemical synthesis, to obtain chimeric proteins. Such proteins might be useful as therapeutic agent in that they may substitute the gene product in individuals with aberrant expression of the GPR50 gene.

The sequence of the gene may also be used in the preparation of vector molecules for the expression of the encoded protein in suitable host cells. A wide variety of host cell and cloning vehicle combinations may be usefully employed in cloning the nucleic acid sequence coding for the GPR50 protein of the invention or parts thereof. For example, useful cloning vehicles may include chromosomal, non-chromosomal and synthetic DNA sequences such as various known bacterial plasmids and wider host range plasmids and vectors derived from combinations of plasmids and phage or virus DNA.

Vehicles for use in expression of the genes or a ligand-binding domain thereof of the present invention will further comprise control sequences operably linked to the nucleic acid sequence coding for a ligand-binding domain. Such control sequences generally comprise a promoter sequence and sequences which regulate and/or enhance expression levels. Of course control and other sequences can vary depending on the host cell selected.

Suitable expression vectors are for example bacterial or yeast plasmids, wide host range plasmids and vectors derived from combinations of plasmid and phage or virus DNA. Vectors derived from chromosomal DNA are also included. Furthermore an origin of replication and/or a dominant selection marker can be present in the vector according to the invention. The vectors according to the invention are suitable for transforming a host cell.

Recombinant expression vectors comprising the DNA of the invention as well as cells transformed with said DNA or said expression vector also form part of the present invention.

Suitable host cells according to the invention are bacterial host cells, yeast and other fungi, plant or animal host such as Chinese Hamster Ovary cells, Human Embryonic Kidney cells or monkey cells. Thus, a host cell which comprises the DNA or expression vector according to the invention is also within the scope of the invention. The engineered host cells can be cultured in conventional nutrient media which can be modified e.g. for appropriate selection, amplification or induction of transcription. The culture conditions such as temperature, pH, nutrients etc. are well known to those ordinary skilled in the art.

The techniques for the preparation of the DNA or the vector according to the invention as well as the transformation or transfection of a host cell with said DNA or vector are standard and well known in the art, see for instance Sambrook et al., Molecular Cloning: A laboratory Manual. 2^{nd} Ed., Cold Spring Harbor Laboratory, Cold Spring Harbor, N.Y., 1989.

The proteins according to the invention can be recovered and purified from recombinant cell cultures by common biochemical purification methods including ammonium sulfate precipitation, extraction, chromatography such as hydrophobic interaction chromatography, cation or anion exchange chromatography or affinity chromatography and high performance liquid chromatography. If necessary, also protein refolding steps can be included.

Another embodiment of the present invention is directed to a method for identifying clinical Bipolar Depression in a human wherein a biological sample containing polynucleotides is obtained from said human, which is analyzed for the presence of a diagnostic polynucleotide, said diagnostic polynucleotide encoding the GPR50 receptor having an A at position 1582 and/or 1804, or an insertion at position 1503-1504 in combination with a G at position 1582 and/or 1804 of SEQ ID NO.: 1 and wherein said gene has been identified as having polymorphism when the presence of said diagnostic polynucleotide is detected in said biological sample.

GPR50 gene products according to the present invention can be used for the in vivo or in vitro identification of novel ligands or analogs thereof. For this purpose e.g. binding studies can be performed with cells transformed with DNA according to the invention or an expression vector comprising DNA according to the invention, said cells expressing the GPR50 gene products according to the invention. Alternatively also the GPR50 gene products itself or ligand-binding domains thereof can be used in an assay for the identification of functional ligands or analogs for the GPR50 gene products. According to the present invention it has been found that GPR50 is associated with BPAD and UP. Thus, compounds binding to GPR50 can be used to modulate the state of these diseases.

Methods to determine binding to expressed gene products as well as in vitro and in vivo assays to determine biological activity of gene products are well known. In general, expressed gene product is contacted with the compound to be tested and binding, stimulation or inhibition of a functional response, such as e.g. signal transduction capacity, is measured.

The following examples are illustrative for the invention and should in no way be interpreted as limiting the scope of the invention.

### EXAMPLES

### Example 1

### PCR Amplification of GPR50

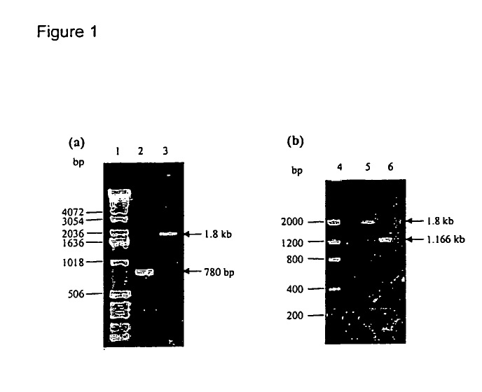

Full and partial cDNA encoding GPR50 were amplified by PCR using proof reading Expand polymerase (Roche), and oligonucleotide primers based upon the sequence of GPR50 shown in SEQ ID NO:1. The template used for the PCR reactions was human 5′-stretch pituitary cDNA library, Marathon-ready human hypothalamus cDNA (Clontech) or human genomic DNA (Promega). Full and partial GPR50 PCR products are shown in FIG. 1. In the case of amplification of full length GPR50, the 5′ primer contained a Hind III site with the following sequence:

5′-GACAAGCTTATGGGGCCCACCCTAGCGGTTCCCACC-3′
(primer 1)
and the 3′ primers each contained a BamHl site
with the following sequences:
5′-CTGGGATCCCACAGCCATTTCATCAGGATC-3′ (no stop
codon for ligation into pcDNA3.1(+) Myc His (B))
(primer 2).
5′-CTGGGATCCTCACACAGCCATTTCATCAGGATC-3′ (with stop
codon for ligation in pcDNA3.1 (+)Hygro)
(primer 3).

The following additional sense primers were used for amplification of partial length GPR50 fragments:

| | |
|---|---|
| 5′-GCCTGTCCTGCTGTGGAGGAAAC-3′ | (primer 4) |
| 5′-ATCCTGACAACCAACTTGCTGAGGTTCGC-3′ | (primer 5) |

The cycling conditions used were as follows:

Following an initial denaturation step at 94° C. for 2 minutes, the reaction was allowed to cycle 33 to 35 times through a sequence of temperatures: 1) denaturation at 94° C. for 30 seconds, 2) primer annealing at 60° C. for 1 minute, 3) elongation at 72° C. for 2 to 3 minutes. A final elongation step at 72° C. for 6 minutes was performed to ensure generation of full length products.

### Example 2

### Cloning of Full Length GPR50

The full length GPR50 cDNA generated in the PCR reaction described above was ligated into the mammalian expression vectors pcDNA3.1/Myc-His-(B) or pcDNA3.1(+) Hygro (Invitrogen). Following chemical transformation and mini-prep DNA isolation, restriction digestion was performed using Hind III and BamH I to identify positive clones.

### Example 3

### Sequencing Analysis of GPR50 from cDNA Sources

DNA sequencing was performed using the ABI prism® BigDye™ Terminator Cycle Sequencing Ready Reaction Kit. Purified PCR products were either sequenced directly, or cloned into the pcDNA3.1/Myc-His or pcDNA3.1Hygro vector, followed by sequencing of individual positive clones. Primers employed in the sequencing reactions included the GPR50 sequence-specific primers, or primers designed to the T7 promoter site and pcDNA3.1/BGH reverse priming site present on the pcDNA3.1 vector. Sequences were compared using DNAMAN program software.

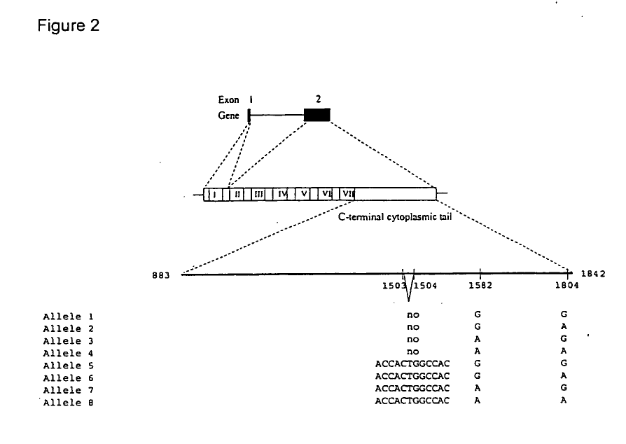

The sequencing of many independent GPR50 clones isolated from pituitary or hypothalamus revealed the existence of several allelic variants for this nucleotide sequence. The seven possible allelic variants for GPR50 are shown in FIG. 2, and all of the variant nucleotides occur in the C-terminal cytoplasmic tail of the translated protein. The allelic variations are located at the positions 1582, 1804 and 1503-1504 of SEQ ID NO: 1. Position 1582 can either be A or G, position 1804 can be either A or G and there is either the presence or absence of a 12 nucleotide insertion at position 1503-1504, consisting of the nucleotide stretch ACC ACT GGC CAC.

Furthermore, sequencing analysis of GPR50 revealed that the published GPR50 cDNA sequence (accession number U52219) contained two sequencing errors. These are located at positions 958 and 1343 on SEQ ID NO:1. Position 958 is C (not T) which changes proline to serine, and position 1343 is C (not G), which changes glycine to alanine.

### Example 4

### Sequencing of GPR50 from Individuals' Genomic DNA

Since several polymorphisms were identified in the GPR50 sequence, genomic DNA was obtained from 14 control patients in order to examine whether individuals contained different sequences for GPR50. Partial PCR products were amplified from each of these samples using the gene-specific (primer 2 and primer 5) and the purified fragments (1166 bp) were sequenced directly. Several individuals were found to contain the GPR50 sequence with the 12 nucleotide insertion, others contained the sequence without the insertion and approximately half contained sequences with and without the insertion. The nucleotides at position 1582 and 1804 were again each variant between A and G. The sequencing results are summarised in Table 1. Since males contain only one copy of the X-chromosome, heterozygous sequences for GPR50 were found only in females. Although a total of eight alleles were possible for GPR50, certain sequences were found to be more prevalent. For example, if the sequence contained the insertion, positions 1594 and 1816 were most often A and G, respectively (allele 7), and if the sequence did not contain the insertion, positions 1582 and 1804 were most often G and A, respectively (allele 2). Moreover, allele 7 was the most common sequence represented in the 14 genomic DNA samples.

### Example 5

### Determination of GPR50 Allelic Variants by Restriction Analysis

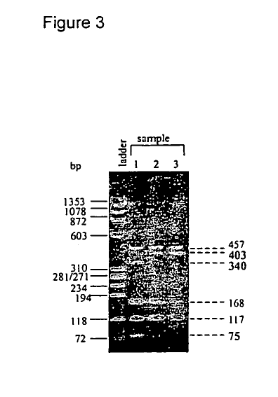

A Bal I restriction endonuclease site was found to be contained within the 12 nucleotide insertion site, as well as at several other sites in the GPR50 sequence. This allowed determination of the GPR50 allelic variants which did or did not contain the insertion. Partial length GPR50 was amplified from individuals' genomic DNA using the primers corresponding to primer 2 and primer 5. The PCR products were purified and 300 ng of each was digested with Bal I at 37° C. for 2 hrs, followed by resolution on 2% agarose gels containing ethidium bromide and visualised under UV illuminescence. Bal I digestion gave rise to the following fragment sizes to indicate the presence or absence of the insertion: Fragments of 340 bp and 75 bp indicated the 12 nucleotide insertion; a fragment of 403 bp indicated no insertion and bands of 403 bp, 340 bp and 75 bp showed that alleles with and without the insertion were both present. FIG. 3 shows Bal I digestion of GPR50 PCR products from samples 1, 2 and 3. This indicates that sample 1 contains only GPR50 allele(s) with the insertion, sample two has alleles with and without the insertion and sample 3 contains only GPR50 sequence(s) with no insertion. This therefore agrees with the sequencing results presented in Table 1.

### Example 6

### Tissue Distribution Analysis of GPR50

The GPR50 cDNA was amplified by PCR using primer 4 (sense) and primer 2 (antisense), which produced a 0.78 kb probe corresponding to the C-terminal region of this receptor. The PCR product was purified and the DNA concentration was estimated by agarose gel electrophoresis. The cDNA (100 ng) was radiolabelled using the High Prime random primer DNA labeling method (Boeringer Mannheim), and the probe was subsequently purified away from unincorporated nucleotides using ProbeQuant G-50 micro columns (Amersham Pharmacia Biotech). Prehybridisation and hybridisation was performed using ExpressHyb solution (clontech) according to the manufacturers guidelines. The MTE array was subjected to a series of washing steps as follows: four 20 min washes at 65° C. in 2×SSC and 1% SDS; and two 20 min washes at 55° C. in 0.1×SSC and 0.5% SDS. All washing steps were performed with continuous agitation. The MTE was wrapped in Saran wrap and exposed to X-ray film with an intensifying screen at −70° C. overnight.

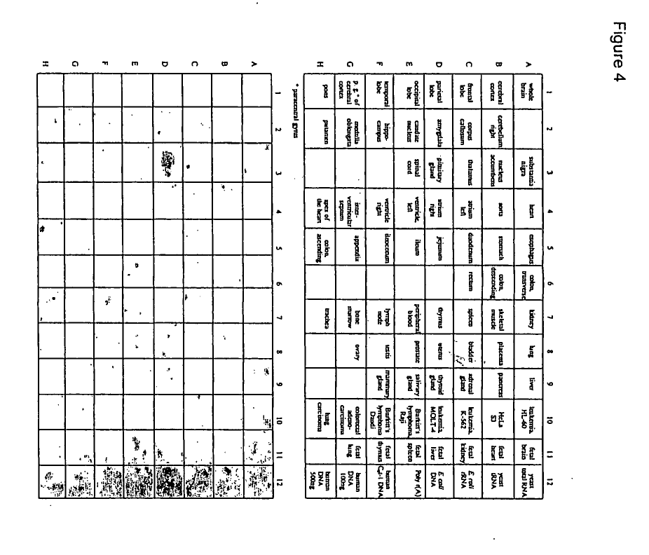

As shown in FIG. 4, a strong hybridising signal was observed only in pituitary. The expression of human GPR50 has previously been reported to be restricted to pituitary and hypothalamus (Reppert et al., 1996), and therefore the results obtained here agree with this data. No expression of GPR50 was detected in any of the peripheral tissues shown in FIG. 4. Expression of GPR50 was confirmed in hypothalamus by PCR (FIG. 1b). Confirmation of GPR50 expression in pituitary and hypothalamus, and failure to detect expression of this transcript in any of the other tissues examined in FIG. 4 support a role for this orphan receptor in HPA axis function.

### Example 7

### Association of GPR50 Polymorphisms with Bipolar Affective Disorder and Recurrent Unipolar Depression

A case-control association study was performed with the 12-nucleotide insertion/deletion polymorphism at position 1503-1504 and the single nucleotide polymorphism, SNP 1804. The insertion/deletion was genotyped in 801 unrelated subjects, including those with diagnoses of bipolar affective disorder (BPAD) (274), recurrent unipolar depression (UP) (262) or schizophrenia (SCZ) (265) and 519 unrelated control subjects. The SNP was genotyped in 777 unrelated subjects, including those with diagnoses of BPAD (257), UP (260) or SCZ (260) and 452 unrelated control subjects. Table 2 shows the number of subjects by sex and diagnosis.

Samples:

854 unrelated subjects, consisting of individuals with diagnoses of bipolar affective disorder (296), recurrent unipolar depression (269) or schizophrenia (289) were inpatients or outpatients of psychiatric services in the South of Scotland. Consensus diagnoses were made according to DSM-IV criteria (Diagnostic and Statistical Manual of Mental Disorders, American Psychiatric Association, Washington D.C., 1994) after personal interview by experienced psychiatrists (Professor Douglas Blackwood, Dr Walter Muir) using the Schedule for Affective Disorders and Schizophrenia—lifetime version (Endicott J, Spitzer R L. 1978. A diagnostic interview: the schedule for affective disorders and schizophrenia. Archives of General Psychiatry 35:837-844 and case note review). 610 control subjects of known age and sex were recruited from the same geographic region and included some subjects recruited by the local Blood Transfusion Service. Control subjects were interviewed using a short questionnaire and had no history of major mental illness.

Standard procedures were used to extract DNA from peripheral blood samples.

Genotyping:

Primers were designed to amplify across the insertion/deletion polymorphism and the SNP. An additional extension primer was designed to genotype the SNP in a SNaPshot™ primer extension reaction.

Insertion/Deletion Polymorphism

| | |
|---|---|
| Primer A: | TTCATTTCAAGCCTGCTTCC |
| Primer B: | CTTAGGGTGGCTGGTAGTGG |

PCR product design size: 185/197

SNP 1804

| | |
|---|---|
| Primer A: | CACTGCTGACTATCCCAAGC |
| Primer B: | TCACACAGCCATTTCATCAG |
| Extension primer: | GATCATCTTCAACATCAA |

SNP: A/G
Genotyping the Insertion/Deletion

PCR reactions for genotyping the insertion/deletion polymorphism were carried out on a PTC225 (MJ Research) using 24 ng total DNA, 10 pmol of each primer, 100 μM dNTPs (Sigma), 1.5 mM MgCl₂ and 1U Taq DNA polymerase (Sigma) in 1×PCR buffer II (Applied Biosystems). The PCR programme used was as follows: an initial denaturation of 94° C. for 3 minutes, followed by 10 cycles of 94° C. for 15 secs, 65° C.-1° C./cycle for 30 secs, and 72° C. for 45 secs.

Samples were diluted and 211 added to 2 μl TAMRA loading buffer containing: 5 vol. deionised formamide: 2 vol. 25 mM EDTA, 50 mg/ml blue dextran, 1 vol. GeneScan®-350 [TAMRA]™ internal lane standard (Applied Biosystems), 1 vol. H₂O. Samples were denatured at 94° C. for 5 minutes and electrophoresis performed on an ABI PRISM 377 DNA Sequencer.

SNP Genotyping

PCR reactions for SNP genotyping were carried out on a PTC225 (MJ Research) using 24 ng total DNA, 2.5 pmol of each primer, 100 μM dNTPs (Sigma), 1.5 mM MgCl₂ and 1U Taq DNA polymerase (Sigma) in 1×PCR buffer II (Applied Biosystems). The PCR programme used was as follows: an initial denaturation of 94° C. for 3 minutes, followed by 10 cycles of 94° C. for 15 secs, 65° C.-1° C./cycle for 30 secs, and 72° C. for 45 secs. PCR primers and dNTPs were removed prior to genotyping: 411 of PCR product were incubated with 1 μl of ExoSapIT (Amersham-Pharmacia) for 45 minutes at 37° C., followed by 20 minutes at 80° C. for enzyme inactivation.

Genotyping reactions were carried out in a final volume of 10 μl containing: 2 μl of cleaned up PCR product, 1 μl SnaPshot™ multiplex mix (Applied Biosystems), 2 pmoles extension primer (designed according to manufacturers recommendations). PCR conditions were 25 cycles of 94° C. for 10 secs, 50° C. for 5 secs, and 60° C. for 30 secs. After cycling unincorporated ddNTPs were removed by adding 1 U of shrimp alkaline phosphatase (Amersham-Pharmacia) and incubating for 45 minutes at 37° C., followed by 20 minutes at 80° C. for enzyme inactivation. 2 μl of loading buffer (5 vol. deionised formamide: 1 vol. 25 mM EDTA, 50 mg/ml blue dextran) were added to 2 μl of SNaPshot™ reaction and the samples denatured at 94° C. for 5 minutes. Electrophoresis was performed on an ABI PRISM 377 DNA Sequencer.

Results were analysed using the GeneScan Analysis Software version 3.1, and for the insertion/deletion polymorphism were further analysed using Genotyper version 1.1.

Statistical Analysis

Association analysis was carried out on the basis of diagnosis and of gender, and at the level of allele frequency, genotype and haplotype. For the association studies as described in this example, when describing the 1503-1504 insertion/deletion polymorphism, allele 1 corresponds to absence of insertion (i.e. deletion) and allele 2 corresponds to the presence of an insertion. Similarly, in reference to SNP 1804, allele 1 corresponds to Adenosine and allele 2 to Guanine. The genotype and haplotype descriptions are described within the appropriate tables.

Prior to statistical analysis being carried out, it was necessary to estimate the effect of genotyping errors and to confirm that the control population constituted a random sample of the population. Firstly, analysis was performed on the genotype frequency and genotyping errors in males. Since GPR50 is located on the X chromosome, heterozygous males were assumed to result from genotyping errors. The error rates were calculated as 1.8% for the insertion/deletion polymorphism and 2.8% for SNP 1804 (average 2.3%). The genotype error rate was assumed the same in females as in males. The error rate as measured by the presence of ‘male’ heterozygotes was considered to be very low and would therefore have no impact on the results. The analysis of male heterozygotes at one or more loci was excluded from further analysis.

Allele frequencies were estimated in the control samples and compared with Hardy-Weinberg (H—W) expectations in the controls (Table 3). The H—W Equilibrium equation uses the formula p²+2pq+q²=1, where p is frequency of allele 1, q is frequency of allele 2 and p+q=1. Therefore p² is probability of genotype 1/1 occurring, q² is probability of genotype 2/2 occurring and 2pq is probability of genotype 1/2 occurring. The frequency of allele 1 in females (pₜ) and in males (pₘ) were derived separately, then used to calculate the overall frequency of allele 1 (weighted mean, p). The calculated weighted mean p values were used to calculate expected frequencies according to Hardy-Weinberg proportions as shown in Table 3. The observed and expected frequencies were then compared in a Chi-squared test to see if the proportions differed. The results demonstrated that the differences between observed and expected frequencies in the control population were not significant and consequently there was no evidence from the H-W test to suggest that there was any bias in the control population. It was therefore believed valid to test these results for association between diagnostic status and the polymorphisms of interest.

Analysis of the allele frequencies in each of the diagnostic groups (Table 4) indicates that there is strong evidence for an association between the deletion polymorphism at position 1503-1504 (allele 1) in BPAD females (p=0.00004) and UP females (p=0.002) (Table 4a). No association was observed with males of these groups or with SCZ (Table 4b). If males and females are combined the resulting p-values are 0.002 and 0.002 for BPAD and UP, respectively, and 0.073 for SCZ (Table 4c).

SNP 1804 shows a similar pattern of association; there is strong evidence for an association between SNP 1804=A (allele 1) in BPAD females (p=0.003) and UP females (0.019) (Table 4a). Again, no association was observed with males or with the SCZ group (Table 4b). If males and females are combined for the SNP, the resulting p-values are 0.003 and 0.032 for BPAD and UP respectively, and 0.022 for SCZ (Table 4c).

If all three diagnostic groups are combined (BPAD, SCZ, and UP) significant association is found with females and the female and male combined group for both the insertion/deletion polymorphism and SNP 1804.

Analysis of the genotype frequencies (Table 5) suggests that genotypes 1/1 are significantly elevated for both the insertion/deletion polymorphism and SNP 1804 in BPAD females (p=0.0002) and UP female (p=0.006). This corresponds to either two copies of GPR50 with the deletion allele or two copies of the allele A at SNP 1804. There is no evidence for association between the genotype for either marker and disease status for males.

Comparison of the haplotype (insertion/deletion and SNP 1804 combined) frequencies in hiales again showed no significant associations (data not shown). Comparison of female haplotypes relies on estimated haplotype frequencies, as the haplotype can not be determined if both markers are heterozygous. The EH program (Terwilliger and Ott, 1994) uses the EM algorithm to assign haplotypes for doubly heterozygous individuals. Analysis of the derived female haplotype frequencies (Table 6), provides evidence for significant association in BPAD, UP and all cases (p=0.0002, p=0.0216 and p=0.0027 respectively).

In summary, in a case-control study designed to assess association between the 12-nucleotide insertion/deletion polymorphism at position 1503-1504 and the single nucleotide polymorphism, SNP 1804, GPR50 was found to be significantly associated with disease status in female BPAD and UP cases, but not in males. This suggests that a GPR50 mutation affects the probability of developing these affective disorders in females or that it is in strong linkage disequilibrium with a mutation which affects that probability.

TABLE 1
Sequencing of GPR50 from individuals' genomic DNA
GPR50 was amplified from genomic DNA from 14 control
individuals, followed by direct sequencing of the purified PCR products.

| Individual | Allele(s) |
|---|---|
| Sample 1 | 7 & 8 |
| Sample 2 | 2 & 7 |
| Sample 3 | 2 |
| Sample 4 | 1 & 7 |
| Sample 5 | 7 |
| Sample 6 | 7 |
| Sample 7 | 1 |
| Sample 8 | 2 |
| Sample 9 | 1 & 7 |
| Sample 10 | 2 |
| Sample 11 | 7 |
| Sample 14 | 2 & 7 |
| Sample 13 | 7 & 8 |
| Sample 14 | 7 |

TABLE 2
Number of individual samples genotyped for each
polymorphism in the association study
All cases include all individuals from the BPAD,
SCZ and UP case groups.

| | Insertion/deletion | | | SNP 1804 | | |
|---|---|---|---|---|---|---|
| | F | M | Total | F | M | Total |
| Control | 226 | 293 | 519 | 198 | 254 | 452 |
| BPAD | 153 | 121 | 274 | 139 | 118 | 257 |
| SCZ | 74 | 191 | 265 | 72 | 188 | 260 |
| UP | 158 | 104 | 262 | 156 | 104 | 260 |
| All cases | 385 | 416 | 801 | 367 | 410 | 777 |
| Total | 611 | 709 | 1320 | 565 | 664 | 1229 |

TABLE 3
Estimating allele frequencies in the controls and comparing
genotype frequencies with Hardy-Weinberg expectations in the controls
For the insertion/deletion polymorphism, deletion is coded as allele 1
and insertion as allele 2; for the SNP 1804, Adenosine is coded as
allele 1 and Guanine as allele 2. The weighted mean p values (allele 1:
0.398 for insertion/deletion and 0.367 for SNP) were used to calculate
expected values according to Hardy-Weinberg proportions.

| | | Insertion/deletion | | | |
|---|---|---|---|---|---|
| | | 1503-1504 | | SNP 1804 | |
| | | No. | No. | No. | No. |
| | Genotype | Observed | Expected | Observed | Expected |
| Females | 1/1 | 21 | 28.8 | 17 | 24.5 |
| N = 182 | 1/2 | 92 | 87.2 | 87 | 84.6 |
| | 2/2 | 69 | 66.1 | 78 | 72.8 |
| Males | 1 | 98 | 92.7 | 92 | 85.5 |
| N = 233 | 2 | 135 | 140.2 | 141 | 147.5 |

TABLE 4
Frequency of allele 1 for insertion/deletion polymorphism
and allele 1 for SNP 1804 in patient and control groups
Allele 1 for insertion/deletion corresponds to deletion and allele 1 for
SNP 1804 corresponds to A. The allele frequencies observed in each
case group were compared to that in the control using a Chi-square
contingency test. The reported p-value results from a Chi-squared
contingency table test (with 1 degree of freedom) that tests the null
hypothesis: Are the allele frequencies equal between
case and control groups.

| Insertion/deletion | | | SNP 1804 | | |
|---|---|---|---|---|---|
| No. chro- | % with | | No. chro- | % with | |
| mosomes | allele1 | P-value | mosomes | allele1 | P-value |

(a) Females

| | | | | | | |
|---|---|---|---|---|---|---|
| Control | 452 | 37.0 | | 396 | 33.1 | |
| BPAD | 306 | 52.0 | 0.00004 | 278 | 44.2 | 0.003 |
| SCZ | 148 | 38.5 | 0.732 | 144 | 39.5 | 0.161 |
| UP | 316 | 48.4 | 0.002 | 312 | 41.7 | 0.019 |
| All cases | 770 | 47.9 | 0.0002 | 734 | 42.2 | 0.003 |

(b) Males

| | | | | | | |
|---|---|---|---|---|---|---|
| Control | 293 | 42.3 | | 254 | 39.4 | |
| BPAD | 121 | 39.2 | 0.619 | 118 | 45.7 | 0.232 |
| SCZ | 191 | 49.7 | 0.109 | 188 | 45.7 | 0.169 |
| UP | 104 | 48.1 | 0.310 | 104 | 43.3 | 0.478 |
| All cases | 416 | 46.3 | 0.283 | 410 | 45.1 | 0.232 |

(c) Males and Females combined

| | | | | | | |
|---|---|---|---|---|---|---|
| Control | 745 | 39.0 | | 650 | 35.5 | |
| BPAD | 427 | 48.3 | 0.002 | 396 | 44.7 | 0.003 |
| SCZ | 339 | 44.8 | 0.073 | 332 | 43.1 | 0.022 |
| UP | 416 | 48.3 | 0.002 | 416 | 42.1 | 0.032 |
| All cases | 1182 | 47.4 | 0.0003 | 1144 | 43.2 | 0.001 |

TABLE 5
Analysis of genotype frequencies in females
Genotype 1/1 for the insertion/deletion polymorphism corresponds to
two copies of the deletion allele, genotype 2/2 corresponds to two
copies of the 12 nucleotide insertion at position 1503-1504, and
genotype 1/2 corresponds to one deletion allele and one insertion allele.
Genotype 1/1 for SNP 1804 corresponds to two copies of GPR50 with
allele A, genotype 2/2 corresponds to two copies of allele G, and
genotype 1/2 corresponds to GPR50 with one A allele and one G allele.
The genotype frequencies observed in each case group were compared
to that in the control using a Chi-square contingency test. The reported
p-value results from a Chi-squared contingency table test (with 2
degrees of freedom) that tests the null hypothesis: Are the genotype
frequencies equal between case and control groups.

| | No. with | No. with | No. with | | |
|---|---|---|---|---|---|
| | genotype | genotype | genotype | | |
| | 1/1 | 1/2 | 2/2 | Total | P-value |
| Insertion/ | | | | | |
| deletion | | | | | |
| Control | 30 | 107 | 89 | 226 | |
| BPAD | 42 | 75 | 36 | 153 | 0.0002 |
| SCZ | 13 | 31 | 30 | 74 | 0.578 |
| UP | 40 | 70 | 46 | 156 | 0.006 |
| All cases | 95 | 176 | 112 | 383 | 0.001 |
| SNP | | | | | |
| 1804 | | | | | |
| Control | 19 | 93 | 86 | 198 | |
| BPAD | 32 | 59 | 48 | 139 | 0.003 |
| SCZ | 10 | 38 | 25 | 73 | 0.329 |
| UP | 29 | 71 | 55 | 155 | 0.035 |
| All cases | 71 | 168 | 128 | 367 | 0.006 |

TABLE 6
Estimated haplotype frequencies in females
Haplotype 1-1 corresponds to deletion and A SNP, 1-2 is deletion and
G SNP, 2-1 is insertion and A SNP and 2-2 is insertion and G SNP.
The EM algorithm in the EH program (Terwilliger & Ott, 1994) was
used to assign haplotypes for doubly heterozygous individuals. The
reported p-value results from a Chi-squared test statistic that tests the
null hypothesis: Are the haplotype frequencies equal between case and
control groups. The chi-squared test statistic (X²⁾ is calculated from
the log likelihoods for the case, control and combined data
X² = 2(In(Lcase) + In(Lcontrol) − In(Lcombined)) (e.g. Sham 1998)

| | | % hap- | % hap- | % hap- | % hap- | |
|---|---|---|---|---|---|---|
| | No. | lotype | lotype | lotype | lotype | X² p- |
| | individuals | 1-1 | 1-2 | 2-1 | 2-2 | value |
| Control | 182 | 23.8 | 13.0 | 9.4 | 53.8 | |
| BPAD | 136 | 31.8 | 19.6 | 13.0 | 35.8 | 0.0002 |
| SCZ | 70 | 30.8 | 7.8 | 8.5 | 52.9 | 0.268 |
| UP | 150 | 30.0 | 17.0 | 11.7 | 41.2 | 0.0216 |
| All cases | 356 | 30.9 | 16.1 | 11.5 | 41.4 | 0.0027 |

Legends to the Figures
FIG. 1 PCR Amplification of GPR50 from Human Pituitary and Human Hypothalamus cDNA.

GPR50 was amplified from a human pituitary cDNA library (a) and human hypothalamus cDNA (b) by PCR using gene-specific primers designed according to SEQ ID NO:1. Lanes 1 and 4 contain the DNA molecular size markers (1 kb ladder and low DNA mass ladder, respectively, Gibco-BRL).

FIG. 2 Allelic Variations of the GPR50 Nucleotide Sequence.

The seven allelic variants (alleles 2-8) for the GPR50 nucleotide sequence are shown. The GPR50 gene is comprised of 2 exons separated by an intron of approximately 3 kb. The TM domains I-VII are indicated, followed by a large C-terminal cytoplasmic tail. All of the variant nucleotides are contained within the C-terminal region. Numbering of nucleotides corresponds to alleles without the 12 nucleotide insertion. Allele 1 is represented by SEQ ID NO: 1, allele 2 (allelic variant) is represented by SEQ ID NO: 2, allele 3 (allelic variant) is represented by SEQ ID NO: 3 and so on to SEQ ID NO: 8.

FIG. 3 Bal I Restriction Analysis of GPR50 to Determine Alleles Containing the 12 Nucleotide Insertion.

GPR50 was amplified from individuals' genomic DNA and the purified PCR products were digested with Bal I, followed by resolution on 2% agarose gels. Fragments of 340 and 75 bp indicated the sequence did contain the insertion; a fragment of 403 bp indicated the sequence did not contain the insertion; and all three of these bands showed that sequences with and without the 12 nucleotide insertion were present.

FIG. 4 Tissue Distribution Analysis of GPR50.

A multiple tissue expression (MTE) array (Clontech) containing Poly A+ RNAs from a wide range of human tissues was probed with a 0.78 kb radiolabelled fragment of GPR50 corresponding to the 3′-end of this cDNA.

FIG. 5 Sequence Alignment of GPR50 Alleles 1 to 8.

Amino acid sequence alignment of the seven GPR50 allelic variants (alleles 2-8). The protein sequence of allele 1 is represented by SEQ ID NO: 9, allele 2 (allelic variant) is represented by SEQ ID NO: 10, allele 3 (allelic variant) is represented by SEQ ID NO: 11 and so on to SEQ ID NO: 16.

### References

Baron, M., Straub, R. E., Lehner, T., Endicott, J., Ott, J., Gilliam, T. C., Lerer, B. (1994) Bipolar disorder and linkage to Xq28. Nature Genet. 7: 461-2.
Civelli, O., Reinscheid, R. K., Nothacker, H. P., and Civelli, O. (1999) Orphan receptors, novel neuropeptides and reverse pharmaceutical research. Brain Res. 848: 63-65.
Conway, S., Drew, J. E., Mowat, E. S., Barrett, P., Delagrange, P. and Morgan, P. J. (2000) Chimeric melatonin mt1 and melatonin-related receptors: Identification of domains and residues participating in ligand binding and receptor activation of the melatonin mt1 receptor. J. Biol. Chem. 275: 20602-9.
Drew, J. E., Barrett, P., Mercer, J. G., Moar, K. M., Canet, E., Delagrange, P., Morgan, P. J. (2001) Localization of the melatonin-related receptor in the rodent brain and peripheral tissues. J Neuroendocrinol. 13: 453-8.
Gubitz, A. K. and Reppert, S. M. (1999) Assignment of the melatonin-related receptor to human chromosome X (GPR50) and mouse chromosome X (Gpr50). Genomics 55: 248-51.
Gubitz, A. K. and Reppert, S. M. (2000) Chimeric and point-mutated receptors reveal that a single glycine residue in transmembrane domain 6 is critical for high affinity melatonin binding. Endocrinol. 141: 1236-1244.
Hinuma, S., Habata, Y., Fujii, R., Kawamata, Y., Hosova, M., Kukusumi, S., Kitada, C. et al (1998) A prolactin-releasing peptide in the brain. Nature, 393: 272-276.
Jackson, A., Iwasiow, R. M. and Tiberi, M. (2000) Distinct function of the cytoplasmic tail in human D1-like receptor ligand binding and coupling. FEBS Lett. 470: 183-188.
Kuhlbrandt W. (2000) Bacteriorhodopsin—the movie. Nature 406: 569-70.
Lefkowitz R. J. (1991) Thrombin receptor. Variations on a theme. Nature 351: 353-354.
Lipman D J, Pearson W R (1985) Rapid and sensitive protein similarity searches. Science, 227: 1435-1441.
Murphy, A. J., Paul, J. I. and Webb, D. R. (1998) From DNA to drugs: the orphan G-protein coupled receptors. Curr. Opin. Drug Discov. Dev. 1: 192-199.
O'Dowd, B. F., Nguyen, T., Marchese, A., Cheng, R., Lynch, K. R., Heng, H. H. Q., Kolakowski, Jr., L. F. and George, S. R. (1997) Discovery of three novel G-protein-coupled receptor genes. Genomics 47: 310-313.
Palczewski, K., Kumasaka, T., Hori, T., Behnke, C. A., Motoshima, H., Fox, B. A., Le Trong, I., Teller; D. C., Okada, T., Stenkamp, R. E., Yamamoto, M., Miyano, M. (2000) Crystal structure of rhodopsin: A G protein-coupled receptor Science 289: 3945.
Probst, W. C., Snyder, L. A., Schuster, D. I., Brosius, J. and Sealfon, S. C. (1992) Sequence alignment of the G-protein coupled receptor superfamily DNA Cell Biol. 11: 1-20.
Reinscheid, R. K., Nothacker, H. and Civelli, O. (2000) The orphanin FQ/nociceptin gene: structure, tissue distribution of expression and functional implications obtained from knockout mice. Peptides, 21: 901-906.
Reppert, S. M. and Weaver, D. R. (1995) Melatonin madness. Cell 83: 1059-1062.
Reppert, S. M., Weaver, D. R., Ebisawa, T., Mahle, C. D. and Kolakowski, L. F. Jr (1996) Cloning of a melatonin-related receptor from human pituitary. FEBS Lett. 386: 219-24.
Saiki R. K., Bugawan T. L., Horn G. T., Mullis K. B. and Erlich H. A. (1986) Analysis of enzymatically amplified beta-globin and HLA-DQ alpha DNA with allele-specific oligonucleotide probes. Nature, 324: 163-166.
Sakurai T, Amemiya A, Ishii M, Matsuzaki I, Chemelli R M, Tanaka H, Williams S C, et al. (1998) Orexins and orexin receptors: a family of hypothalammic neuropeptides and G protein-coupled receptors that regulate feeding behavior. Cell, 92: 573-585.
Sham, P (1998). Statistics in Human Genetics. Arnold, London. P162.
Smith and Waterman (1981) Identification of common molecular subsequences. J. Mol. Biol., 147: 195-197.
Stine, O. C. McMahon, F. C., Chen, L., Xu, J., Meyers, D. A., MacKinnon, D. F., Simpson, S. et al (1997) Initial genome screen for bipolar disorder in the NIMH genetics initiative pedigrees: chromosomes 2, 11, 13, 14, and X. Am J Med Genet. 74: 263-269.
Strader, C. D., Fong, T. M., Graziano, M. P. and Tota, M. R. (1995) The family of G-protein-coupled receptors. FASEB J. 9: 745-754.
Terwilliger J D and Ott J (1994) Handbook of Human Linkage Analysis. Baltimore: John Hopkins University Press.
Tiruppathi C, Yan W, Sandoval R, Naqvi T, Pronin A N, Benovic J L and Malik A B. (2000) G protein-coupled receptor kinase-5 regulates thrombin-activated signalling in endothelial cells. PNAS 97: 7440-7445.
Trapaidze, N., Cvejic, S., Nivarthi, R. N., Abood, M. and Devi, L. A. (2000) Role for C-tail residues in delta opioid receptor downregulation. DNA Cell Biol. 19: 93-101.
Tsao, P. and von Zastrow, M. (2000) Downregulation of G protein-coupled receptors, Curr. Opin. Neurobiol. 10: 365-369.
Wang, J., Wang, L., Zheng, J., Anderson, J. L. and Toews, M. L. (2000) Identification of distinct carboxyl-terminal domains mediating internalization and down-regulation of the hamster alpha(1B)-adrenergic receptor. Mol. Pharmacol. 57: 687-94.
Wess, J. (1997). G-protein-coupled receptors: molecular mechanisms involved in receptor activation and sensitivity of G-protein recognition. FASEB J. 11: 346-354.
Wilson, S., Bergsma, D. J., Chambers, J. K., Muir, A. I., Fantom, K. G., Ellis, C., Murdock, P. R., et al. (1998) Orphan G-protein-coupled receptors: the next generation of drug targets? Br J. Pharmacol. 125: 1387-92.

1. An isolated polynucleotide encoding a GPR50 receptor protein, wherein said polynucleotide has at least one polymorphic site.

2. The isolated polynucleotide of claim 1, wherein said polymorphic site is localised at position 1503-1504, 1582 or 1804 of SEQ ID NO.: 1.

3. The isolated polynucleotide of claim 1, wherein said polynucleotide comprises any one of SEQ ID NO.: 2 to 8.

4. The isolated polynucleotide of claim 1, wherein said polynucleotide encodes a polypeptide comprising any one of SEQ ID NO.: 10 to 16.

5. The isolated polynucleotide of claim 1, wherein said polynucleotide has an A at position 1582 and/or 1804, or an insertion at position 1503-1504 in combination with a G at position 1582 and/or 1804.

6. A recombinant expression vector comprising the polynucleotide according to claim 1.

7. Polypeptide encoded by the polynucleotide according to claim 1.

8. A cell transfected with the polynucleotide according to claim 1.

9. The cell according to claim 8 which is a stable transfected cell which expresses the polypeptide according to claim 7.

10. (canceled)

11. (canceled)

12. (canceled)

13. A method for determining binding of ligands of GPR50 protein or a polymorphic variant thereof according to claim 7, to prepare a medicament for a psychiatric disorder, preferably BPAD or UP, said method comprising the steps of:
a) introducing into a suitable host cell a polynucleotide according to any of claims 1-5 or a recombinant expression vector according to claim 6;
b) culturing the host cells under conditions to allow expression of the introduced polynucleotide;
c) optionally isolating the expression product;
bringing the expression product from step c or the host cell from step b into contact with potential ligands;
establishing the amount of binding of the ligand to the expressed protein or its signal transduction capacity; and optionally,
isolating the ligand.

14. A method for the formulation of a pharmaceutical composition comprising the method of claims 14 and mixing the compound identified with a pharmaceutically acceptable carrier.

15. A method for identifying an increased risk for clinical Bipolar Depression or UP in a human comprising analyzing a biological sample of an individual for the presence of a polynucleotide, said polynucleotide encoding the GPR50 receptor having an A at position 1804, and/or an absence of an insertion at position 1503-1504 of SEQ ID NO.: 1; and
identifying said gene as having polymorphism associated with BPAD or UP when the presence of said polynucleotide is detected in said biological sample.

16. A diagnostic assay for detection of a psychiatric disorder comprising the steps of detecting mutations in the nucleic acid sequences encoding the gene of claim 1.

17. The assay of claim 16 wherein the step of detecting mutations is performed by a method selected from the group consisting of PCR, specific hybridization, and detection of relative levels of RNA.

18. The assay of claim 16 wherein the step of detecting mutations is detecting the presence and/or the levels of the GPR50.

19. The assay of claim 18 wherein the step of detecting the presence and/or levels of the GPR50 is performed by a method selected from the group consisting of immunological technologies and binding of specific antibodies.


SEQUENCE LISTING (26 sequences)

SEQ ID NO: 1 - DNA, 1842 bp, Homo sapiens
atggggcccaccctagcggttcccaccccctatggctgtattggctgtaagctaccccagccagaatacccaccggctctaatcatctttatgttctgcgcgatggttatcaccatcgttgtagacctaatcggcaactccatggtcattttggctgtgacgaagaacaagaagctccggaattctggcaacatcttcgtggtcagtctctctgtggccgatatgctggtggccatctacccataccctttgatgctgcatgccatgtccattgggggctgggatctgagccagttacagtgccagatggtcgggttcatcacagggctgagtgtggtcggctccatcttcaacatcgtggcaatcgctatcaaccgttactgctacatctgccacagcctccagtacgaacggatcttcagtgtgcgcaatacctgcatctacctggtcatcacctggatcatgaccgtcctggctgtcctgcccaacatgtacattggcaccatcgagtacgatcctcgcacctacacctgcatcttcaactatctgaacaaccctgtcttcactgttaccatcgtctgcatccacttcgtcctccctctcctcatcgtgggtttctgctacgtgaggatctggaccaaagtgctggcggcccgtgaccctgcagggcagaatcctgacaaccaacttgctgaggttcgcaattttctaaccatgtttgtgatcttcctcctctttgcagtgtgctggtgccctatcaacgtgctcactgtcttggtggctgtcagtccgaaggagatggcaggcaagatccccaactggctttatcttgcagcctacttcatagcctacttcaacagctgcctcaacgctgtgatctacgggctcctcaatgagaatttccgaagagaatactggaccatcttccatgctatgcggcaccctatcatattcttctctggcctcatcagtgatattcgtgagatgcaggaggcccgtaccctggcccgcgcccgtgcccatgctcgcgaccaagctcgtgaacaagaccgtgcccatgcctgtcctgctgtggaggaaaccccgatgaatgtccggaatgttccattacctggtgatgctgcagctggccaccccgaccgtgcctctggccaccctaagccccattccagatcctcctctgcctatcgcaaatctgcctctacccaccacaagtctgtctttagccactccaaggctgcctctggtcacctcaagcctgtctctggccactccaagcctgcctctggtcaccccaagtctgccactgtctaccctaagcctgcctctgtccatttcaaggctgactctgtccatttcaagggtgactctgtccatttcaagcctgactctgttcatttcaagcctgcttccagcaaccccaagcccatcactggccaccatgtctctgctggcagccactccaagtctgccttcagtgctgccaccagccaccctaaacccatcaagccagctaccagccatgctgagcccaccactgctgactatcccaagcctgccactaccagccaccctaagcccgctgctgctgacaaccctgagctctctgcctcccattgccccgagatccctgccattgcccaccctgtgtctgacgacagtgacctccctgagtcggcctctagccctgccgctgggcccaccaagcctgctgccagccagctggagtctgacaccatcgctgaccttcctgaccctactgtagtcactaccagtaccaatgattaccatgatgtcgtggttgttgatgttgaagatgatcctgatgaaatggctgtgtga

SEQ ID NO: 2 - DNA, 1842 bp, Homo sapiens
atggggcccaccctagcggttcccaccccctatggctgtattggctgtaagctaccccagccagaatacccaccggctctaatcatctttatgttctgcgcgatggttatcaccatcgttgtagacctaatcggcaactccatggtcattttggctgtgacgaagaacaagaagctccggaattctggcaacatcttcgtggtcagtctctctgtggccgatatgctggtggccatctacccataccctttgatgctgcatgccatgtccattgggggctgggatctgagccagttacagtgccagatggtcgggttcatcacagggctgagtgtggtcggctccatcttcaacatcgtggcaatcgctatcaaccgttactgctacatctgccacagcctccagtacgaacggatcttcagtgtgcgcaatacctgcatctacctggtcatcacctggatcatgaccgtcctggctgtcctgcccaacatgtacattggcaccatcgagtacgatcctcgcacctacacctgcatcttcaactatctgaacaaccctgtcttcactgttaccatcgtctgcatccacttcgtcctccctctcctcatcgtgggtttctgctacgtgaggatctggaccaaagtgctggcggcccgtgaccctgcagggcagaatcctgacaaccaacttgctgaggttcgcaattttctaaccatgtttgtgatcttcctcctctttgcagtgtgctggtgccctatcaacgtgctcactgtcttggtggctgtcagtccgaaggagatggcaggcaagatccccaactggctttatcttgcagcctacttcatagcctacttcaacagctgcctcaacgctgtgatctacgggctcctcaatgagaatttccgaagagaatactggaccatcttccatgctatgcggcaccctatcatattcttctctggcctcatcagtgatattcgtgagatgcaggaggcccgtaccctggcccgcgcccgtgcccatgctcgcgaccaagctcgtgaacaagaccgtgcccatgcctgtcctgctgtggaggaaaccccgatgaatgtccggaatgttccattacctggtgatgctgcagctggccaccccgaccgtgcctctggccaccctaagccccattccagatcctcctctgcctatcgcaaatctgcctctacccaccacaagtctgtctttagccactccaaggctgcctctggtcacctcaagcctgtctctggccactccaagcctgcctctggtcaccccaagtctgccactgtctaccctaagcctgcctctgtccatttcaaggctgactctgtccatttcaagggtgactctgtccatttcaagcctgactctgttcatttcaagcctgcttccagcaaccccaagcccatcactggccaccatgtctctgctggcagccactccaagtctgccttcagtgctgccaccagccaccctaaacccatcaagccagctaccagccatgctgagcccaccactgctgactatcccaagcctgccactaccagccaccctaagcccgctgctgctgacaaccctgagctctctgcctcccattgccccgagatccctgccattgcccaccctgtgtctgacgacagtgacctccctgagtcggcctctagccctgccgctgggcccaccaagcctgctgccagccagctggagtctgacaccatcgctgaccttcctgaccctactgtagtcactaccagtaccaatgattaccatgatgtcgtggttattgatgttgaagatgatcctgatgaaatggctgtgtga

SEQ ID NO: 3 - DNA, 1842 bp, Homo sapiens
atggggcccaccctagcggttcccaccccctatggctgtattggctgtaagctaccccagccagaatacccaccggctctaatcatctttatgttctgcgcgatggttatcaccatcgttgtagacctaatcggcaactccatggtcattttggctgtgacgaagaacaagaagctccggaattctggcaacatcttcgtggtcagtctctctgtggccgatatgctggtggccatctacccataccctttgatgctgcatgccatgtccattgggggctgggatctgagccagttacagtgccagatggtcgggttcatcacagggctgagtgtggtcggctccatcttcaacatcgtggcaatcgctatcaaccgttactgctacatctgccacagcctccagtacgaacggatcttcagtgtgcgcaatacctgcatctacctggtcatcacctggatcatgaccgtcctggctgtcctgcccaacatgtacattggcaccatcgagtacgatcctcgcacctacacctgcatcttcaactatctgaacaaccctgtcttcactgttaccatcgtctgcatccacttcgtcctccctctcctcatcgtgggtttctgctacgtgaggatctggaccaaagtgctggcggcccgtgaccctgcagggcagaatcctgacaaccaacttgctgaggttcgcaattttctaaccatgtttgtgatcttcctcctctttgcagtgtgctggtgccctatcaacgtgctcactgtcttggtggctgtcagtccgaaggagatggcaggcaagatccccaactggctttatcttgcagcctacttcatagcctacttcaacagctgcctcaacgctgtgatctacgggctcctcaatgagaatttccgaagagaatactggaccatcttccatgctatgcggcaccctatcatattcttctctggcctcatcagtgatattcgtgagatgcaggaggcccgtaccctggcccgcgcccgtgcccatgctcgcgaccaagctcgtgaacaagaccgtgcccatgcctgtcctgctgtggaggaaaccccgatgaatgtccggaatgttccattacctggtgatgctgcagctggccaccccgaccgtgcctctggccaccctaagccccattccagatcctcctctgcctatcgcaaatctgcctctacccaccacaagtctgtctttagccactccaaggctgcctctggtcacctcaagcctgtctctggccactccaagcctgcctctggtcaccccaagtctgccactgtctaccctaagcctgcctctgtccatttcaaggctgactctgtccatttcaagggtgactctgtccatttcaagcctgactctgttcatttcaagcctgcttccagcaaccccaagcccatcactggccaccatgtctctgctggcagccactccaagtctgccttcagtgctgccaccagccaccctaaacccatcaagccagctaccagccatgctgagcccaccactgctgactatcccaagcctgccactaccagccaccctaagcccactgctgctgacaaccctgagctctctgcctcccattgccccgagatccctgccattgcccaccctgtgtctgacgacagtgacctccctgagtcggcctctagccctgccgctgggcccaccaagcctgctgccagccagctggagtctgacaccatcgctgaccttcctgaccctactgtagtcactaccagtaccaatgattaccatgatgtcgtggttgttgatgttgaagatgatcctgatgaaatggctgtgtga

SEQ ID NO: 4 - DNA, 1842 bp, Homo sapiens
atggggcccaccctagcggttcccaccccctatggctgtattggctgtaagctaccccagccagaatacccaccggctctaatcatctttatgttctgcgcgatggttatcaccatcgttgtagacctaatcggcaactccatggtcattttggctgtgacgaagaacaagaagctccggaattctggcaacatcttcgtggtcagtctctctgtggccgatatgctggtggccatctacccataccctttgatgctgcatgccatgtccattgggggctgggatctgagccagttacagtgccagatggtcgggttcatcacagggctgagtgtggtcggctccatcttcaacatcgtggcaatcgctatcaaccgttactgctacatctgccacagcctccagtacgaacggatcttcagtgtgcgcaatacctgcatctacctggtcatcacctggatcatgaccgtcctggctgtcctgcccaacatgtacattggcaccatcgagtacgatcctcgcacctacacctgcatcttcaactatctgaacaaccctgtcttcactgttaccatcgtctgcatccacttcgtcctccctctcctcatcgtgggtttctgctacgtgaggatctggaccaaagtgctggcggcccgtgaccctgcagggcagaatcctgacaaccaacttgctgaggttcgcaattttctaaccatgtttgtgatcttcctcctctttgcagtgtgctggtgccctatcaacgtgctcactgtcttggtggctgtcagtccgaaggagatggcaggcaagatccccaactggctttatcttgcagcctacttcatagcctacttcaacagctgcctcaacgctgtgatctacgggctcctcaatgagaatttccgaagagaatactggaccatcttccatgctatgcggcaccctatcatattcttctctggcctcatcagtgatattcgtgagatgcaggaggcccgtaccctggcccgcgcccgtgcccatgctcgcgaccaagctcgtgaacaagaccgtgcccatgcctgtcctgctgtggaggaaaccccgatgaatgtccggaatgttccattacctggtgatgctgcagctggccaccccgaccgtgcctctggccaccctaagccccattccagatcctcctctgcctatcgcaaatctgcctctacccaccacaagtctgtctttagccactccaaggctgcctctggtcacctcaagcctgtctctggccactccaagcctgcctctggtcaccccaagtctgccactgtctaccctaagcctgcctctgtccatttcaaggctgactctgtccatttcaagggtgactctgtccatttcaagcctgactctgttcatttcaagcctgcttccagcaaccccaagcccatcactggccaccatgtctctgctggcagccactccaagtctgccttcagtgctgccaccagccaccctaaacccatcaagccagctaccagccatgctgagcccaccactgctgactatcccaagcctgccactaccagccaccctaagcccactgctgctgacaaccctgagctctctgcctcccattgccccgagatccctgccattgcccaccctgtgtctgacgacagtgacctccctgagtcggcctctagccctgccgctgggcccaccaagcctgctgccagccagctggagtctgacaccatcgctgaccttcctgaccctactgtagtcactaccagtaccaatgattaccatgatgtcgtggttattgatgttgaagatgatcctgatgaaatggctgtgtga

SEQ ID NO: 5 - DNA, 1854 bp, Homo sapiens
atggggcccaccctagcggttcccaccccctatggctgtattggctgtaagctaccccagccagaatacccaccggctctaatcatctttatgttctgcgcgatggttatcaccatcgttgtagacctaatcggcaactccatggtcattttggctgtgacgaagaacaagaagctccggaattctggcaacatcttcgtggtcagtctctctgtggccgatatgctggtggccatctacccataccctttgatgctgcatgccatgtccattgggggctgggatctgagccagttacagtgccagatggtcgggttcatcacagggctgagtgtggtcggctccatcttcaacatcgtggcaatcgctatcaaccgttactgctacatctgccacagcctccagtacgaacggatcttcagtgtgcgcaatacctgcatctacctggtcatcacctggatcatgaccgtcctggctgtcctgcccaacatgtacattggcaccatcgagtacgatcctcgcacctacacctgcatcttcaactatctgaacaaccctgtcttcactgttaccatcgtctgcatccacttcgtcctccctctcctcatcgtgggtttctgctacgtgaggatctggaccaaagtgctggcggcccgtgaccctgcagggcagaatcctgacaaccaacttgctgaggttcgcaattttctaaccatgtttgtgatcttcctcctctttgcagtgtgctggtgccctatcaacgtgctcactgtcttggtggctgtcagtccgaaggagatggcaggcaagatccccaactggctttatcttgcagcctacttcatagcctacttcaacagctgcctcaacgctgtgatctacgggctcctcaatgagaatttccgaagagaatactggaccatcttccatgctatgcggcaccctatcatattcttctctggcctcatcagtgatattcgtgagatgcaggaggcccgtaccctggcccgcgcccgtgcccatgctcgcgaccaagctcgtgaacaagaccgtgcccatgcctgtcctgctgtggaggaaaccccgatgaatgtccggaatgttccattacctggtgatgctgcagctggccaccccgaccgtgcctctggccaccctaagccccattccagatcctcctctgcctatcgcaaatctgcctctacccaccacaagtctgtctttagccactccaaggctgcctctggtcacctcaagcctgtctctggccactccaagcctgcctctggtcaccccaagtctgccactgtctaccctaagcctgcctctgtccatttcaaggctgactctgtccatttcaagggtgactctgtccatttcaagcctgactctgttcatttcaagcctgcttccagcaaccccaagcccatcactggccaccatgtctctgctggcagccactccaagtctgccttcagtgctgccaccagccaccctaaacccaccactggccacatcaagccagctaccagccatgctgagcccaccactgctgactatcccaagcctgccactaccagccaccctaagcccgctgctgctgacaaccctgagctctctgcctcccattgccccgagatccctgccattgcccaccctgtgtctgacgacagtgacctccctgagtcggcctctagccctgccgctgggcccaccaagcctgctgccagccagctggagtctgacaccatcgctgaccttcctgaccctactgtagtcactaccagtaccaatgattaccatgatgtcgtggttgttgatgttgaagatgatcctgatgaaatggctgtgtga

SEQ ID NO: 6 - DNA, 1854 bp, Homo sapiens
atggggcccaccctagcggttcccaccccctatggctgtattggctgtaagctaccccagccagaatacccaccggctctaatcatctttatgttctgcgcgatggttatcaccatcgttgtagacctaatcggcaactccatggtcattttggctgtgacgaagaacaagaagctccggaattctggcaacatcttcgtggtcagtctctctgtggccgatatgctggtggccatctacccataccctttgatgctgcatgccatgtccattgggggctgggatctgagccagttacagtgccagatggtcgggttcatcacagggctgagtgtggtcggctccatcttcaacatcgtggcaatcgctatcaaccgttactgctacatctgccacagcctccagtacgaacggatcttcagtgtgcgcaatacctgcatctacctggtcatcacctggatcatgaccgtcctggctgtcctgcccaacatgtacattggcaccatcgagtacgatcctcgcacctacacctgcatcttcaactatctgaacaaccctgtcttcactgttaccatcgtctgcatccacttcgtcctccctctcctcatcgtgggtttctgctacgtgaggatctggaccaaagtgctggcggcccgtgaccctgcagggcagaatcctgacaaccaacttgctgaggttcgcaattttctaaccatgtttgtgatcttcctcctctttgcagtgtgctggtgccctatcaacgtgctcactgtcttggtggctgtcagtccgaaggagatggcaggcaagatccccaactggctttatcttgcagcctacttcatagcctacttcaacagctgcctcaacgctgtgatctacgggctcctcaatgagaatttccgaagagaatactggaccatcttccatgctatgcggcaccctatcatattcttctctggcctcatcagtgatattcgtgagatgcaggaggcccgtaccctggcccgcgcccgtgcccatgctcgcgaccaagctcgtgaacaagaccgtgcccatgcctgtcctgctgtggaggaaaccccgatgaatgtccggaatgttccattacctggtgatgctgcagctggccaccccgaccgtgcctctggccaccctaagccccattccagatcctcctctgcctatcgcaaatctgcctctacccaccacaagtctgtctttagccactccaaggctgcctctggtcacctcaagcctgtctctggccactccaagcctgcctctggtcaccccaagtctgccactgtctaccctaagcctgcctctgtccatttcaaggctgactctgtccatttcaagggtgactctgtccatttcaagcctgactctgttcatttcaagcctgcttccagcaaccccaagcccatcactggccaccatgtctctgctggcagccactccaagtctgccttcagtgctgccaccagccaccctaaacccaccactggccacatcaagccagctaccagccatgctgagcccaccactgctgactatcccaagcctgccactaccagccaccctaagcccgctgctgctgacaaccctgagctctctgcctcccattgccccgagatccctgccattgcccaccctgtgtctgacgacagtgacctccctgagtcggcctctagccctgccgctgggcccaccaagcctgctgccagccagctggagtctgacaccatcgctgaccttcctgaccctactgtagtcactaccagtaccaatgattaccatgatgtcgtggttattgatgttgaagatgatcctgatgaaatggctgtgtga

SEQ ID NO: 7 - DNA, 1854 bp, Homo sapiens
atggggcccaccctagcggttcccaccccctatggctgtattggctgtaagctaccccagccagaatacccaccggctctaatcatctttatgttctgcgcgatggttatcaccatcgttgtagacctaatcggcaactccatggtcattttggctgtgacgaagaacaagaagctccggaattctggcaacatcttcgtggtcagtctctctgtggccgatatgctggtggccatctacccataccctttgatgctgcatgccatgtccattgggggctgggatctgagccagttacagtgccagatggtcgggttcatcacagggctgagtgtggtcggctccatcttcaacatcgtggcaatcgctatcaaccgttactgctacatctgccacagcctccagtacgaacggatcttcagtgtgcgcaatacctgcatctacctggtcatcacctggatcatgaccgtcctggctgtcctgcccaacatgtacattggcaccatcgagtacgatcctcgcacctacacctgcatcttcaactatctgaacaaccctgtcttcactgttaccatcgtctgcatccacttcgtcctccctctcctcatcgtgggtttctgctacgtgaggatctggaccaaagtgctggcggcccgtgaccctgcagggcagaatcctgacaaccaacttgctgaggttcgcaattttctaaccatgtttgtgatcttcctcctctttgcagtgtgctggtgccctatcaacgtgctcactgtcttggtggctgtcagtccgaaggagatggcaggcaagatccccaactggctttatcttgcagcctacttcatagcctacttcaacagctgcctcaacgctgtgatctacgggctcctcaatgagaatttccgaagagaatactggaccatcttccatgctatgcggcaccctatcatattcttctctggcctcatcagtgatattcgtgagatgcaggaggcccgtaccctggcccgcgcccgtgcccatgctcgcgaccaagctcgtgaacaagaccgtgcccatgcctgtcctgctgtggaggaaaccccgatgaatgtccggaatgttccattacctggtgatgctgcagctggccaccccgaccgtgcctctggccaccctaagccccattccagatcctcctctgcctatcgcaaatctgcctctacccaccacaagtctgtctttagccactccaaggctgcctctggtcacctcaagcctgtctctggccactccaagcctgcctctggtcaccccaagtctgccactgtctaccctaagcctgcctctgtccatttcaaggctgactctgtccatttcaagggtgactctgtccatttcaagcctgactctgttcatttcaagcctgcttccagcaaccccaagcccatcactggccaccatgtctctgctggcagccactccaagtctgccttcagtgctgccaccagccaccctaaacccaccactggccacatcaagccagctaccagccatgctgagcccaccactgctgactatcccaagcctgccactaccagccaccctaagcccactgctgctgacaaccctgagctctctgcctcccattgccccgagatccctgccattgcccaccctgtgtctgacgacagtgacctccctgagtcggcctctagccctgccgctgggcccaccaagcctgctgccagccagctggagtctgacaccatcgctgaccttcctgaccctactgtagtcactaccagtaccaatgattaccatgatgtcgtggttgttgatgttgaagatgatcctgatgaaatggctgtgtga

SEQ ID NO: 8 - DNA, 1854 bp, Homo sapiens
atggggcccaccctagcggttcccaccccctatggctgtattggctgtaagctaccccagccagaatacccaccggctctaatcatctttatgttctgcgcgatggttatcaccatcgttgtagacctaatcggcaactccatggtcattttggctgtgacgaagaacaagaagctccggaattctggcaacatcttcgtggtcagtctctctgtggccgatatgctggtggccatctacccataccctttgatgctgcatgccatgtccattgggggctgggatctgagccagttacagtgccagatggtcgggttcatcacagggctgagtgtggtcggctccatcttcaacatcgtggcaatcgctatcaaccgttactgctacatctgccacagcctccagtacgaacggatcttcagtgtgcgcaatacctgcatctacctggtcatcacctggatcatgaccgtcctggctgtcctgcccaacatgtacattggcaccatcgagtacgatcctcgcacctacacctgcatcttcaactatctgaacaaccctgtcttcactgttaccatcgtctgcatccacttcgtcctccctctcctcatcgtgggtttctgctacgtgaggatctggaccaaagtgctggcggcccgtgaccctgcagggcagaatcctgacaaccaacttgctgaggttcgcaattttctaaccatgtttgtgatcttcctcctctttgcagtgtgctggtgccctatcaacgtgctcactgtcttggtggctgtcagtccgaaggagatggcaggcaagatccccaactggctttatcttgcagcctacttcatagcctacttcaacagctgcctcaacgctgtgatctacgggctcctcaatgagaatttccgaagagaatactggaccatcttccatgctatgcggcaccctatcatattcttctctggcctcatcagtgatattcgtgagatgcaggaggcccgtaccctggcccgcgcccgtgcccatgctcgcgaccaagctcgtgaacaagaccgtgcccatgcctgtcctgctgtggaggaaaccccgatgaatgtccggaatgttccattacctggtgatgctgcagctggccaccccgaccgtgcctctggccaccctaagccccattccagatcctcctctgcctatcgcaaatctgcctctacccaccacaagtctgtctttagccactccaaggctgcctctggtcacctcaagcctgtctctggccactccaagcctgcctctggtcaccccaagtctgccactgtctaccctaagcctgcctctgtccatttcaaggctgactctgtccatttcaagggtgactctgtccatttcaagcctgactctgttcatttcaagcctgcttccagcaaccccaagcccatcactggccaccatgtctctgctggcagccactccaagtctgccttcagtgctgccaccagccaccctaaacccaccactggccacatcaagccagctaccagccatgctgagcccaccactgctgactatcccaagcctgccactaccagccaccctaagcccactgctgctgacaaccctgagctctctgcctcccattgccccgagatccctgccattgcccaccctgtgtctgacgacagtgacctccctgagtcggcctctagccctgccgctgggcccaccaagcctgctgccagccagctggagtctgacaccatcgctgaccttcctgaccctactgtagtcactaccagtaccaatgattaccatgatgtcgtggttattgatgttgaagatgatcctgatgaaatggctgtgtga

SEQ ID NO: 9 - protein, 613 aa, Homo sapiens
MGPTLAVPTPYGCIGCKLPQPEYPPALIIFMFCAMVITIVVDLIGNSMVILAVTKNKKLRNSGNIFVVSLSVADMLVAIYPYPLMLHAMSIGGWDLSQLQCQMVGFITGLSVVGSIFNIVAIAINRYCYICHSLQYERIFSVRNTCIYLVITWIMTVLAVLPNMYIGTIEYDPRTYTCIFNYLNNPVFTVTIVCIHFVLPLLIVGFCYVRIWTKVLAARDPAGQNPDNQLAEVRNFLTMFVIFLLFAVCWCPINVLTVLVAVSPKEMAGKIPNWLYLAAYFIAYFNSCLNAVIYGLLNENFRREYWTIFHAMRHPIIFFSGLISDIREMQEARTLARARAHARDQAREQDRAHACPAVEETPMNVRNVPLPGDAAAGHPDRASGHPKPHSRSSSAYRKSASTHHKSVFSHSKAASGHLKPVSGHSKPASGHPKSATVYPKPASVHFKADSVHFKGDSVHFKPDSVHFKPASSNPKPITGHHVSAGSHSKSAFSAATSHPKPIKPATSHAEPTTADYPKPATTSHPKPAAADNPELSASHCPEIPAIAHPVSDDSDLPESASSPAAGPTKPAASQLESDTIADLPDPTVVTTSTNDYHDVVVVDVEDDPDEMAV

SEQ ID NO: 10 - protein, 613 aa, Homo sapiens [sequence omitted]

SEQ ID NO: 11 - protein, 613 aa, Homo sapiens
MGPTLAVPTPYGCIGCKLPQPEYPPALIIFMFCAMVITIVVDLIGNSMVILAVTKNKKLRNSGNIFVVSLSVADMLVAIYPYPLMLHAMSIGGWDLSQLQCQMVGFITGLSVVGSIFNIVAIAINRYCYICHSLQYERIFSVRNTCIYLVITWIMTVLAVLPNMYIGTIEYDPRTYTCIFNYLNNPVFTVTIVCIHFVLPLLIVGFCYVRIWTKVLAARDPAGQNPDNQLAEVRNFLTMFVIFLLFAVCWCPINVLTVLVAVSPKEMAGKIPNWLYLAAYFIAYFNSCLNAVIYGLLNENFRREYWTIFHAMRHPIIFFSGLISDIREMQEARTLARARAHARDQAREQDRAHACPAVEETPMNVRNVPLPGDAAAGHPDRASGHPKPHSRSSSAYRKSASTHHKSVFSHSKAASGHLKPVSGHSKPASGHPKSATVYPKPASVHFKADSVHFKGDSVHFKPDSVHFKPASSNPKPITGHHVSAGSHSKSAFSAATSHPKPIKPATSHAEPTTADYPKPATTSHPKPTAADNPELSASHCPEIPAIAHPVSDDSDLPESASSPAAGPTKPAASQLESDTIADLPDPTVVTTSTNDYHDVVVVDVEDDPDEMAV

SEQ ID NO: 12 - protein, 613 aa, Homo sapiens
MGPTLAVPTPYGCIGCKLPQPEYPPALIIFMFCAMVITIVVDLIGNSMVILAVTKNKKLRNSGNIFVVSLSVADMLVAIYPYPLMLHAMSIGGWDLSQLQCQMVGFITGLSVVGSIFNIVAIAINRYCYICHSLQYERIFSVRNTCIYLVITWIMTVLAVLPNMYIGTIEYDPRTYTCIFNYLNNPVFTVTIVCIHFVLPLLIVGFCYVRIWTKVLAARDPAGQNPDNQLAEVRNFLTMFVIFLLFAVCWCPINVLTVLVAVSPKEMAGKIPNWLYLAAYFIAYFNSCLNAVIYGLLNENFRREYWTIFHAMRHPIIFFSGLISDIREMQEARTLARARAHARDQAREQDRAHACPAVEETPMNVRNVPLPGDAAAGHPDRASGHPKPHSRSSSAYRKSASTHHKSVFSHSKAASGHLKPVSGHSKPASGHPKSATVYPKPASVHFKADSVHFKGDSVHFKPDSVHFKPASSNPKPITGHHVSAGSHSKSAFSAATSHPKPIKPATSHAEPTTADYPKPATTSHPKPTAADNPELSASHCPEIPAIAHPVSDDSDLPESASSPAAGPTKPAASQLESDTIADLPDPTVVTTSTNDYHDVVVIDVEDDPDEMAV

SEQ ID NO: 13 - protein, 617 aa, Homo sapiens
MGPTLAVPTPYGCIGCKLPQPEYPPALIIFMFCAMVITIVVDLIGNSMVILAVTKNKKLRNSGNIFVVSLSVADMLVAIYPYPLMLHAMSIGGWDLSQLQCQMVGFITGLSVVGSIFNIVAIAINRYCYICHSLQYERIFSVRNTCIYLVITWIMTVLAVLPNMYIGTIEYDPRTYTCIFNYLNNPVFTVTIVCIHFVLPLLIVGFCYVRIWTKVLAARDPAGQNPDNQLAEVRNFLTMFVIFLLFAVCWCPINVLTVLVAVSPKEMAGKIPNWLYLAAYFIAYFNSCLNAVIYGLLNENFRREYWTIFHAMRHPIIFFSGLISDIREMQEARTLARARAHARDQAREQDRAHACPAVEETPMNVRNVPLPGDAAAGHPDRASGHPKPHSRSSSAYRKSASTHHKSVFSHSKAASGHLKPVSGHSKPASGHPKSATVYPKPASVHFKADSVHFKGDSVHFKPDSVHFKPASSNPKPITGHHVSAGSHSKSAFSAATSHPKPTTGHIKPATSHAEPTTADYPKPATTSHPKPAAADNPELSASHCPEIPAIAHPVSDDSDLPESASSPAAGPTKPAASQLESDTIADLPDPTVVTTSTNDYHDVVVVDVEDDPDEMAV

SEQ ID NO: 14 - protein, 617 aa, Homo sapiens [sequence omitted]

SEQ ID NO: 15 - protein, 617 aa, Homo sapiens [sequence omitted]

SEQ ID NO: 16 - protein, 617 aa, Homo sapiens
MGPTLAVPTPYGCIGCKLPQPEYPPALIIFMFCAMVITIVVDLIGNSMVILAVTKNKKLRNSGNIFVVSLSVADMLVAIYPYPLMLHAMSIGGWDLSQLQCQMVGFITGLSVVGSIFNIVAIAINRYCYICHSLQYERIFSVRNTCIYLVITWIMTVLAVLPNMYIGTIEYDPRTYTCIFNYLNNPVFTVTIVCIHFVLPLLIVGFCYVRIWTKVLAARDPAGQNPDNQLAEVRNFLTMFVIFLLFAVCWCPINVLTVLVAVSPKEMAGKIPNWLYLAAYFIAYFNSCLNAVIYGLLNENFRREYWTIFHAMRHPIIFFSGLISDIREMQEARTLARARAHARDQAREQDRAHACPAVEETPMNVRNVPLPGDAAAGHPDRASGHPKPHSRSSSAYRKSASTHHKSVFSHSKAASGHLKPVSGHSKPASGHPKSATVYPKPASVHFKADSVHFKGDSVHFKPDSVHFKPASSNPKPITGHHVSAGSHSKSAFSAATSHPKPTTGHIKPATSHAEPTTADYPKPATTSHPKPTAADNPELSASHCPEIPAIAHPVSDDSDLPESASSPAAGPTKPAASQLESDTIADLPDPTVVTTSTNDYHDVVVIDVEDDPDEMAV

SEQ ID NO: 17 - DNA, 36 bp, Artificial
gacaagcttatggggcccaccctagcggttcccacc

SEQ ID NO: 18 - DNA, 30 bp, Artificial
ctgggatcccacagccatttcatcaggatc

SEQ ID NO: 19 - DNA, 33 bp, Artificial
ctgggatcctcacacagccatttcatcaggatc

SEQ ID NO: 20 - DNA, 23 bp, Artificial
gcctgtcctgctgtggaggaaac

SEQ ID NO: 21 - DNA, 29 bp, Artificial
atcctgacaaccaacttgctgaggttcgc

SEQ ID NO: 22 - DNA, 20 bp, Artificial
ttcatttcaagcctgcttcc

SEQ ID NO: 23 - DNA, 20 bp, Artificial
cttagggtggctggtagtgg

SEQ ID NO: 24 - DNA, 20 bp, Artificial
cactgctgactatcccaagc

SEQ ID NO: 25 - DNA, 20 bp, Artificial
tcacacagccatttcatcag

SEQ ID NO: 26 - DNA, 18 bp, Artificial
gatcatcttcaacatcaa